SEQUENCE LISTING (3 sequences)

SEQ ID NO: 1 - DNA, 16 bp, Artificial Sequence
  Synthetic oligonucleotide
ctagtaaatgcttgtc

SEQ ID NO: 2 - DNA, 16 bp, Artificial Sequence
  Synthetic oligonucleotide
gtaatattcagaccag

SEQ ID NO: 3 - DNA, 16 bp, Artificial Sequence
  Synthetic oligonucleotide
ctttattcaatgtggc

(19) United States
(12) Patent Application Publication
(10) Pub. No.: US 2020/0300865 A1
(43) Pub. Date: Sep. 24, 2020

(54) BIOMARKER OF PNPLA3 EXPRESSION

(71) Applicant: AstraZeneca AB, Sodertalje (SE)

(72) Inventor: Mathias LILJEBLAD, Södertälje (SE)

(21) Appl. No.: 16/718,740

(22) Filed: Dec. 18, 2019

Related U.S. Application Data
(60) Provisional application No. 62/781,904, filed on Dec. 19, 2018.

(51) Int. Cl. G01N 33/68; G01N 33/92

(52) U.S. Cl. CPC G01N 33/6893; A61K 45/06; G01N 2800/085; G01N 33/92

(57) ABSTRACT

The present disclosure relates to a novel biomarker for measuring PNPLA3 gene expression and methods of using the novel biomarker for treating, preventing, or ameliorating a disease associated with PNPLA3.

### SEQUENCE LISTING

The present application is being filed along with a Sequence Listing in electronic format. The Sequence Listing is provided as a file entitled 200834-US-PSP SEQUENCE LISTING_ST25.txt created Dec. 19, 2018, which is 1 kb in size. The information in the electronic format of the sequence listing is incorporated herein by reference in its entirety.

### DESCRIPTION

The present disclosure relates to a novel biomarker for measuring gene expression and methods of using the novel biomarker. At least one embodiment of the present disclosure includes a novel biomarker for measuring PNPLA3 (patatin like phospholipase domain containing 3; hypothetical protein dJ796I17.1; adiponutrin; DJ796I17.1) expression and methods of using the biomarker, which can be useful for treating, preventing, or ameliorating a disease associated with PNPLA3.

PNPLA3 is a 481-amino acid member of the patatin-like phospholipase domain-containing family that is expressed in the ER and on lipid droplets. In humans, PNPLA3 is highly expressed in liver hepatocytes, whereas adipose tissue expression is five-fold less (Huang et al, Proc. Natl. Acad. Sci. USA 2010. 107: 7892-7). PNPLA3 is not a circulating protein and because it targets hepatocytes and there is no suitable surrogate tissue to monitor target engagement, there remains a lack of mechanistically derived target engagement biomarker suitable for determining protein expression and efficacy. Thus, there is a clear need for a biomarker that will measure target engagement, and for monitoring the response to treatment with a compound targeting a PNPLA3 nucleic acid.

Applicants have found that the ratio of cholesteryl palmitoleate and cholesteryl palmitate (CE 16:1/16:0) in the plasma or liver of a patient (“the CE 16:1/16:0 ratio”) represents a novel biomarker that may be used for predicting, diagnosing and/or prognosticating a disease associated with PNPLA3. The CE 16:1/16:0 ratio is modulated by stearoyl-CoA desaturase-1 (“SCD1”) activity. SCD1 is an enzyme that actively regulates de novo lipogenesis, PARα activity, polyunsaturated fatty acids and cholesterol levels. (Lee et al, Am J Clin Nutr. 2015, 101(1): 34-43; Chong et al, Am J Clin Nutr. 2008, 87(4):817-23; Miller et al, PNAS 1996, 93 (18) 9443-48; Oosterveer et al. J. Biol. Chem. 2009; 284:34036-44; Ntambi, J. Lipid Res. 1999. 40: 1549-58; Kim et al, J. Lipid Res. 2002, 43, 1750-57). In particular, SCD1 is the enzyme that converts the fatty acid cholesteryl palmitate 16:0 to the fatty acid 16:1 cholesteryl palmitate, so a shift in the CE 16:1/16:0 ratio is likely due to change in SCD1 activity. Moreover, a reduction in PNPLA3 reduces SCD1 activity which subsequently leads to a reduction in the CE 16:1/16:0 ratio incorporated in cholesteryl esters. It is therefore likely that PNPLA3 liver levels regulates SCD1 activity via one or several of these pathways.

Accordingly, the present disclosure relates to the biomarker CE 16:1/16:0 ratio for measuring expression of PNPLA3 and to methods of using the CE 16:1/16:0 ratio in the treatment of a disease associated with PNPLA3. Moreover, the novel biomarker will allow for dose adjustment of a compound targeting a PNPLA3 nucleic acid used in the treatment of a disease associated with PNPLA3, which may allow for optimal treatment of patients being treated for a disease associated with PNPLA3.

A biomarker can be described as “a characteristic that is objectively measured and evaluated as an indicator of normal biologic processes, pathogenic processes, or pharmacologic responses to a therapeutic intervention.” (Wagner et al., Clin. Pharmacol. Ther. 2015; 98(1): 2-5). A biomarker is any identifiable and measurable indicator associated with a particular condition or disease where there is a correlation between the presence or level of the biomarker and some aspect of the condition or disease (including the presence of, the level or changing level of, the type of, the stage of, the susceptibility to the condition or disease, or the responsiveness to a drug used for treating the condition or disease). The correlation may be qualitative, quantitative, or both qualitative and quantitative. In some instances, biomarkers may be used to predict or detect the presence, level, type or stage of conditions or diseases, the susceptibility to conditions or diseases, or the response to treatments. It is thought that biomarkers will play an increasingly important role in the future of drug discovery and development, by improving the efficiency of research and development programs. Biomarkers can be used as diagnostic agents, monitors of disease progression, monitors of treatment and predictors of clinical outcome. For example, various biomarker research projects are attempting to identify markers of specific cancers and of specific cardiovascular and immunological diseases. It is believed that the development of new validated biomarkers will lead both to significant reductions in healthcare and drug development costs and to significant improvements in treatment for a wide variety of diseases and conditions.

In at least one embodiment of the present disclosure, the CE 16:1/16:0 ratio is measured in the plasma of the patient as a biomarker for the expression of PNPLA3. For example, a population of individuals having type 2 diabetes and NAFLD has been identified as having an average CE 16:1/16:0 ratio of 0.3. (Eriksson et al. Diabetologia 2018; 61: 1923-1934). In at least one embodiment, a reduction in average CE 16:1/16:0 ratio below 0.3 indicates a corresponding reduction in PNPLA3 expression.

In other embodiments, the ratio of cholesteryl palmitoleate and cholesteryl palmitate (CE 16:1/16:0) can be used for the screening and/or diagnosis of a disease associated with PNPLA3. For example, a measurement of average CE 16:1/16:0 ratio above 0.3 indicates a patient in need of therapy with a compound associated with the treatment of a disease associated with PNPLA3. In yet another example, a reduction in average CE 16:1/16:0 ratio below 0.3 indicates a patient who has responded to a compound associated with the treatment of a disease associated with PNPLA3. In at least one embodiment, the disease is chosen from liver damage, steatosis, liver fibrosis, liver inflammation, liver scarring or cirrhosis, liver failure, liver enlargement, elevated transaminases, or hepatic fat accumulation in a patient having, or at risk of having, a liver disease associated with PNPLA3. In certain embodiments, the disease is NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis.

In other embodiments, the ratio of cholesteryl palmitoleate and cholesteryl palmitate (CE 16:1/16:0) can be used for monitoring the activity and/or progression of a disease associated with PNPLA3. For example, a measurement of average CE 16:1/16:0 ratio above 0.3 indicates a patient in need of further therapy with a compound associated with the treatment of a disease associated with PNPLA3. In yet another example, a reduction in average CE 16:1/16:0 ratio below 0.3 indicates a patient who has responded to a compound associated with the treatment of a disease associated with PNPLA3. In at least one embodiment, the disease is chosen from liver damage, steatosis, liver fibrosis, liver inflammation, liver scarring or cirrhosis, liver failure, liver enlargement, elevated transaminases, or hepatic fat accumulation in a patient having, or at risk of having, a liver disease associated with PNPLA3. In certain embodiments, the disease is NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis.

In other embodiments, the ratio can be used for the prediction of the efficacy of a compound in a method for the treatment of a disease associated with PNPLA3. In at least one embodiment, the disease is chosen from liver damage, steatosis, liver fibrosis, liver inflammation, liver scarring or cirrhosis, liver failure, liver enlargement, elevated transaminases, or hepatic fat accumulation in a patient having, or at risk of having, a liver disease associated with PNPLA3. In certain embodiments, the disease is NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. In at least one embodiment, a reduction in average CE 16:1/16:0 ratio below 0.3 indicates a corresponding reduction in PNPLA3 expression.

In yet another embodiment, the ratio of cholesteryl palmitoleate and cholesteryl palmitate (CE 16:1/16:0) can be used for adjusting the dose of a compound targeting a PNPLA3 nucleic acid used in the treatment of a disease associated with PNPLA3. In at least one embodiment, the disease is chosen from liver damage, steatosis, liver fibrosis, liver inflammation, liver scarring or cirrhosis, liver failure, liver enlargement, elevated transaminases, or hepatic fat accumulation in a patient having, or at risk of having, a liver disease associated with PNPLA3. In certain embodiments, the disease is NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. In at least one embodiment of the present disclosure, the dose of the compound targeting a PNPLA3 nucleic acid will be increased. In another embodiment, the dose of the compound targeting a PNPLA3 nucleic acid will be decreased.

The diagnostic methods of the specification can be undertaken using a sample previously taken from the patient or patient. Such samples may be preserved by freezing or fixed and embedded in formalin-paraffin or other media. Alternatively, a fresh sample containing sample may be obtained and used.

Certain embodiments provide methods of monitoring activity and/or progression of a disease associated with PNPLA3 where the patient is being treated with a compound targeted to a PNPLA3 nucleic acid. In certain embodiments, a compound comprises or consists of ION 916333 or salt thereof, having the following chemical structure:

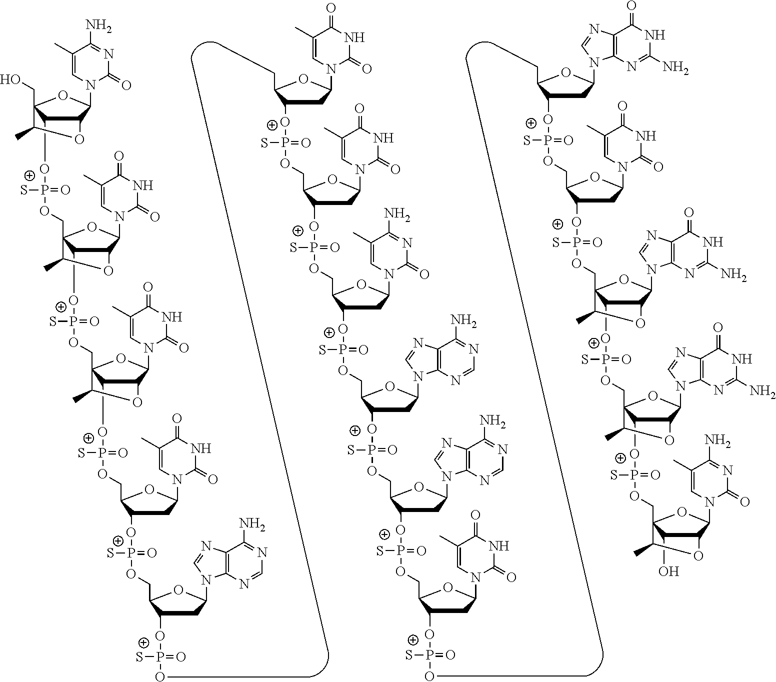

In certain embodiments, a compound comprises or consists of ION 975616 or salt thereof, having the following chemical structure:

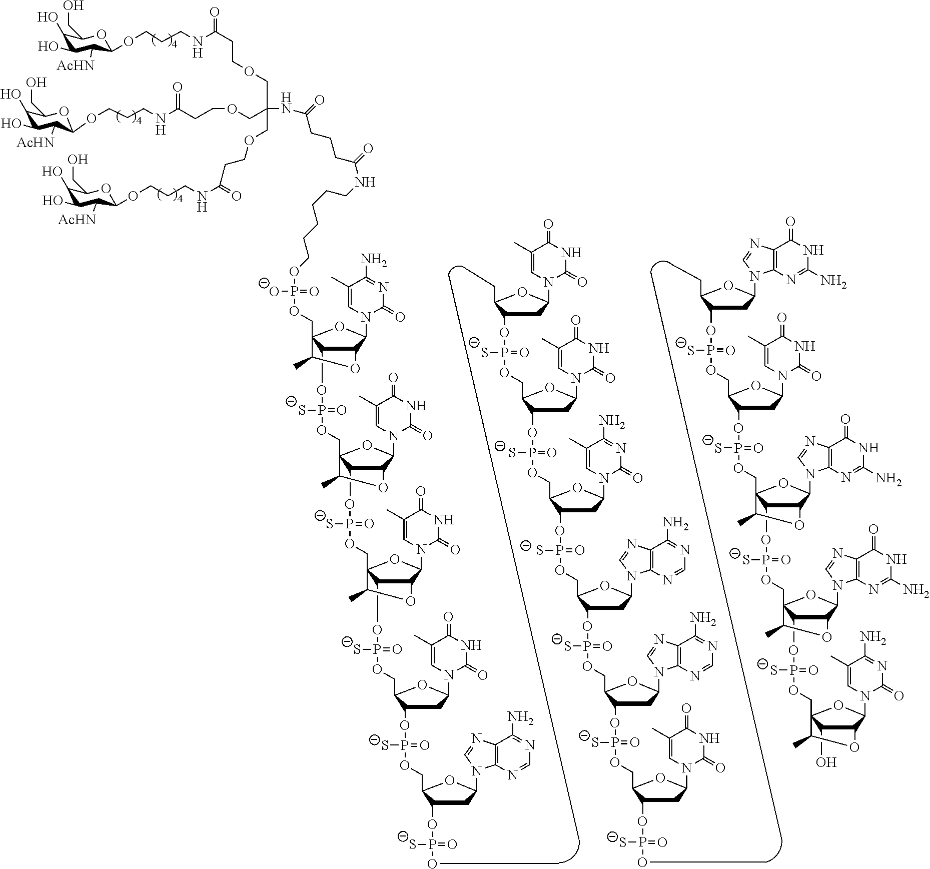

In certain embodiments, a compound comprises or consists of the sodium salt of 975616, having the following chemical structure:

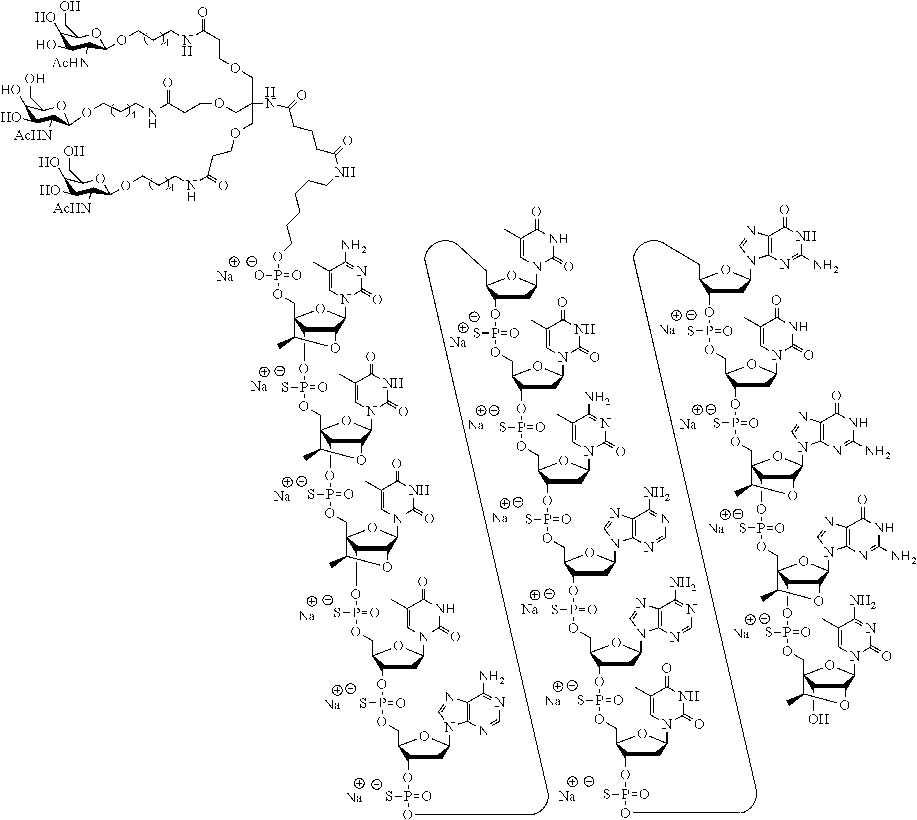

In certain embodiments, a compound comprises or consists of ION 975613 or salt thereof, having the following chemical structure:

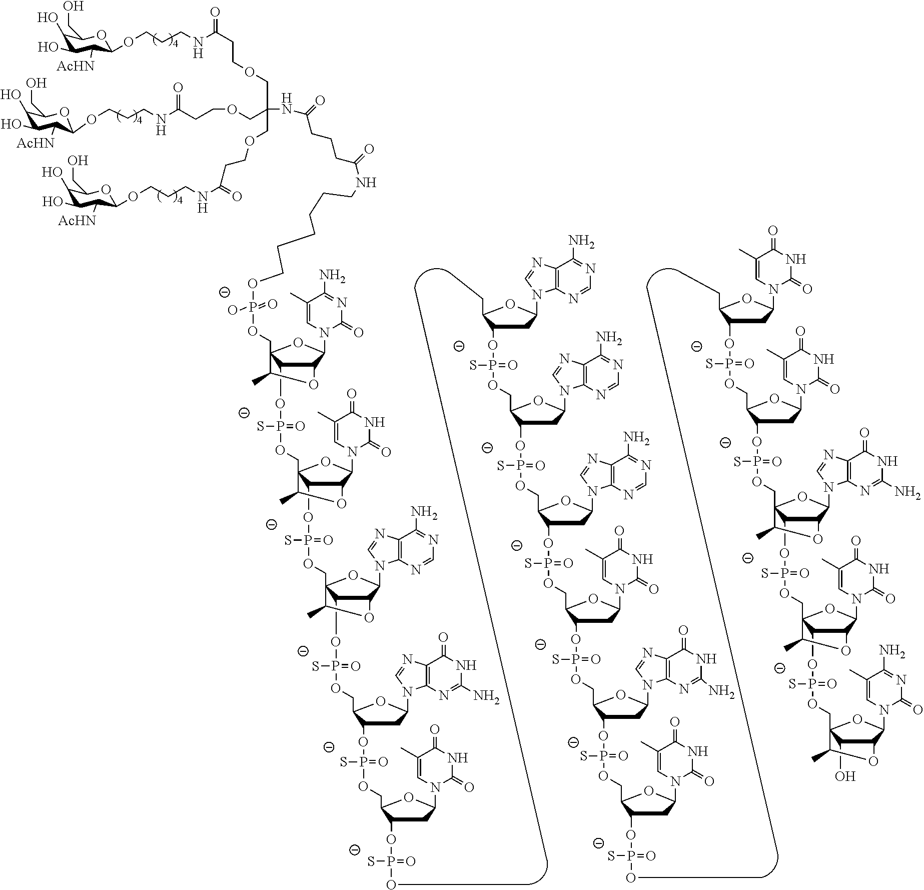

In certain embodiments, a compound comprises or consists of the sodium salt of 975613, having the following chemical structure:

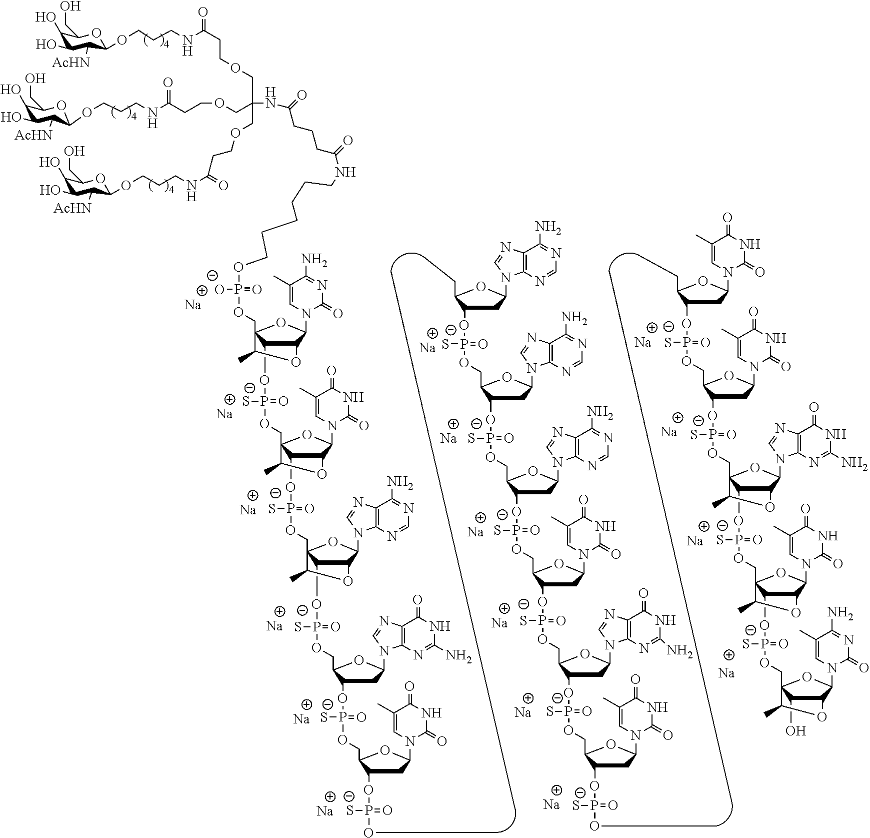

In certain embodiments, a compound comprises or consists of ION 975612 or salt thereof, having the following chemical structure:

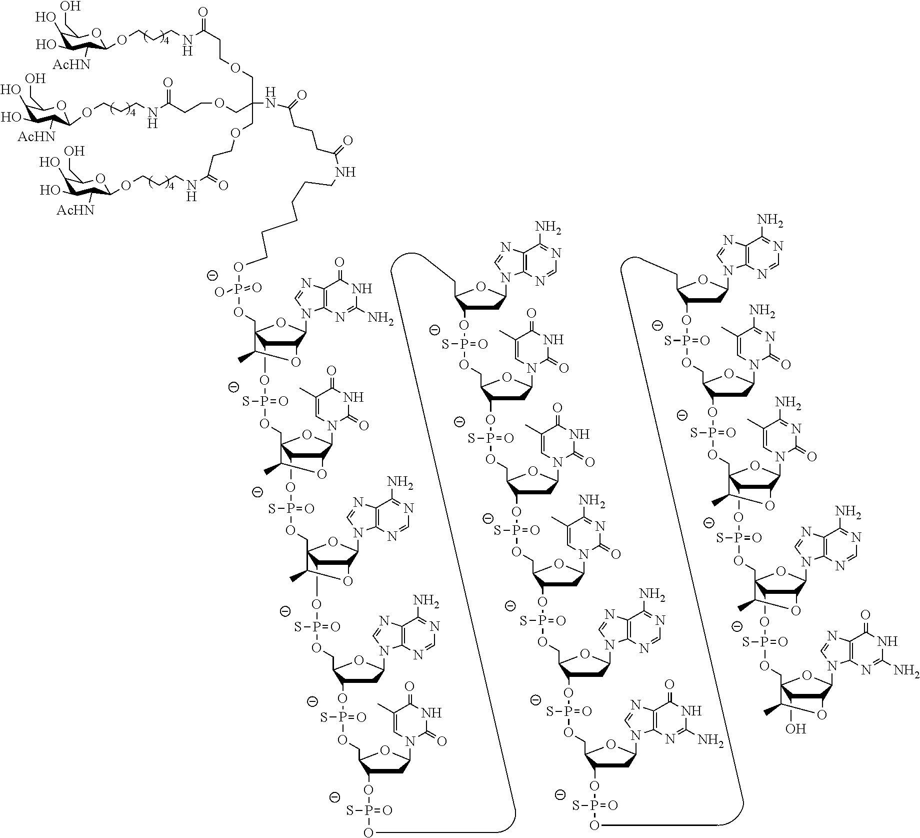

In certain embodiments, a compound comprises or consists of the sodium salt of 975612, having the following chemical structure:

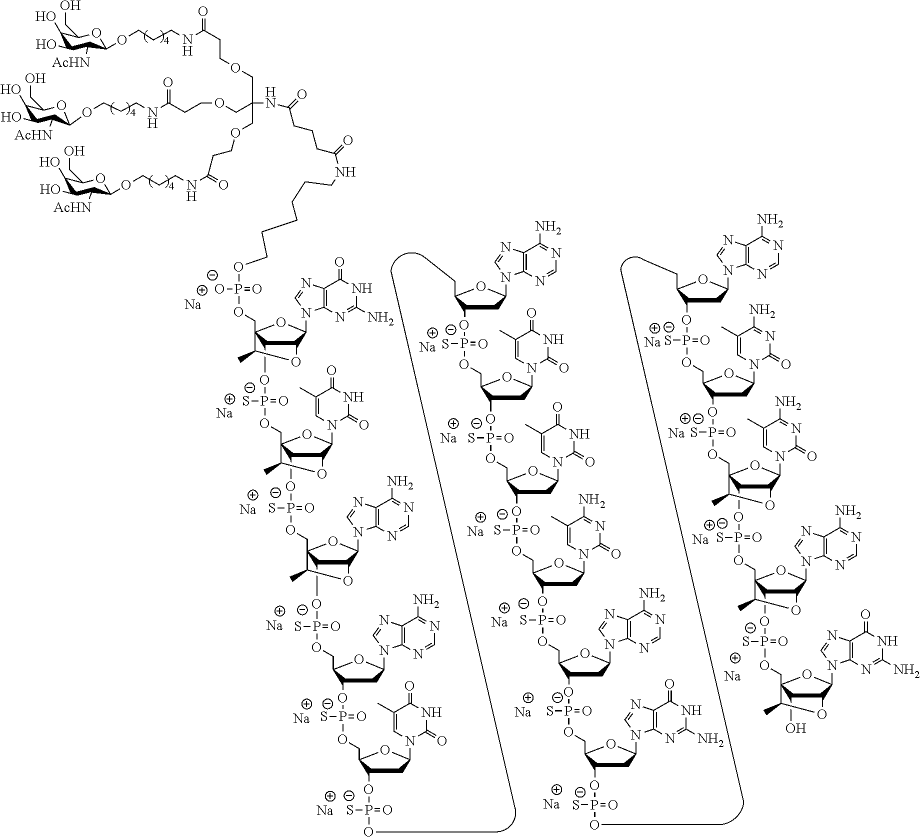

In certain embodiments, a compound comprises or consists of ION 916789 or salt thereof, having the following chemical structure:

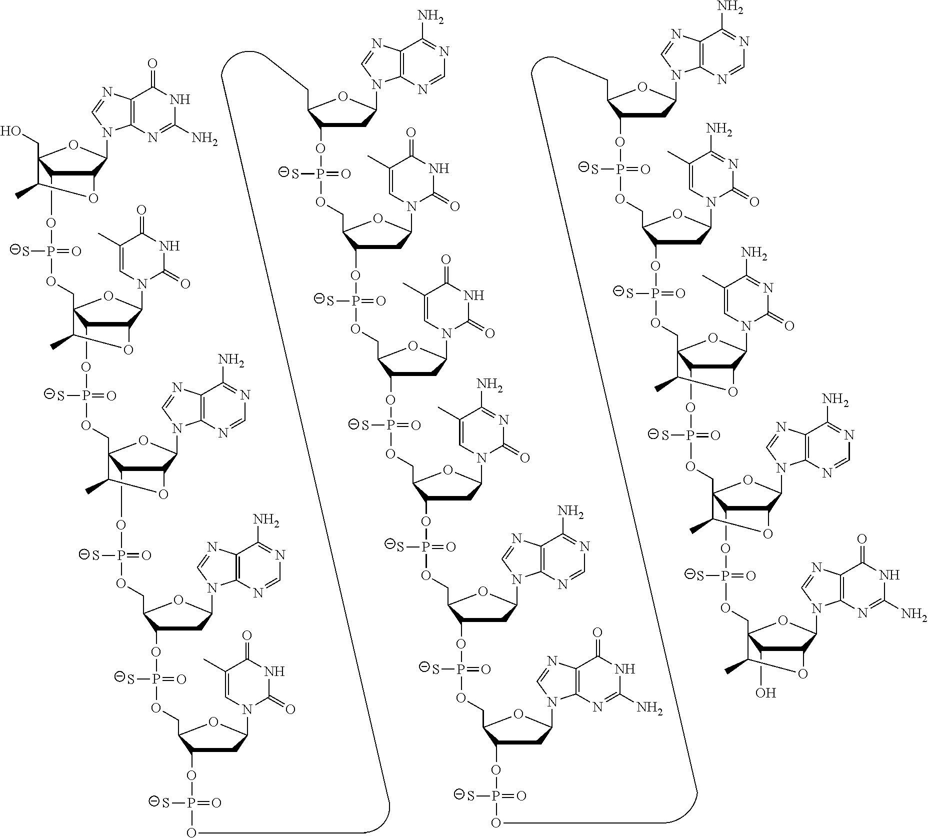

In certain embodiments, a compound comprises or consists of the sodium salt of 916789, having the following chemical structure:

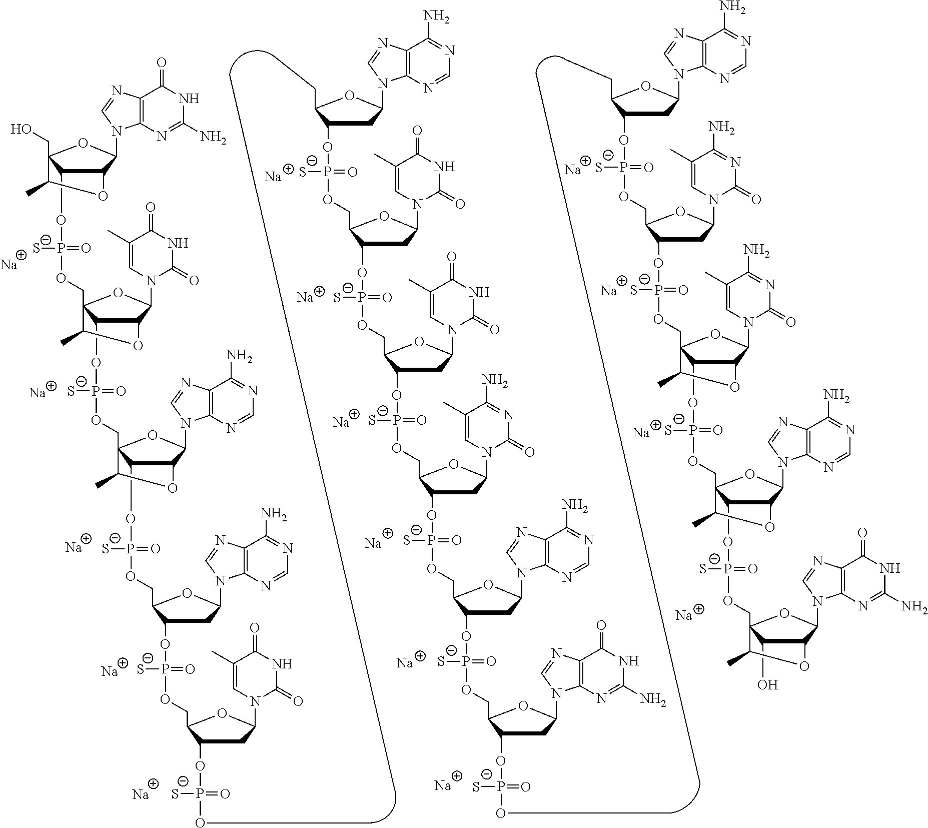

In certain embodiments, a compound comprises or consists of ION 916602 or salt thereof, having the following chemical structure:

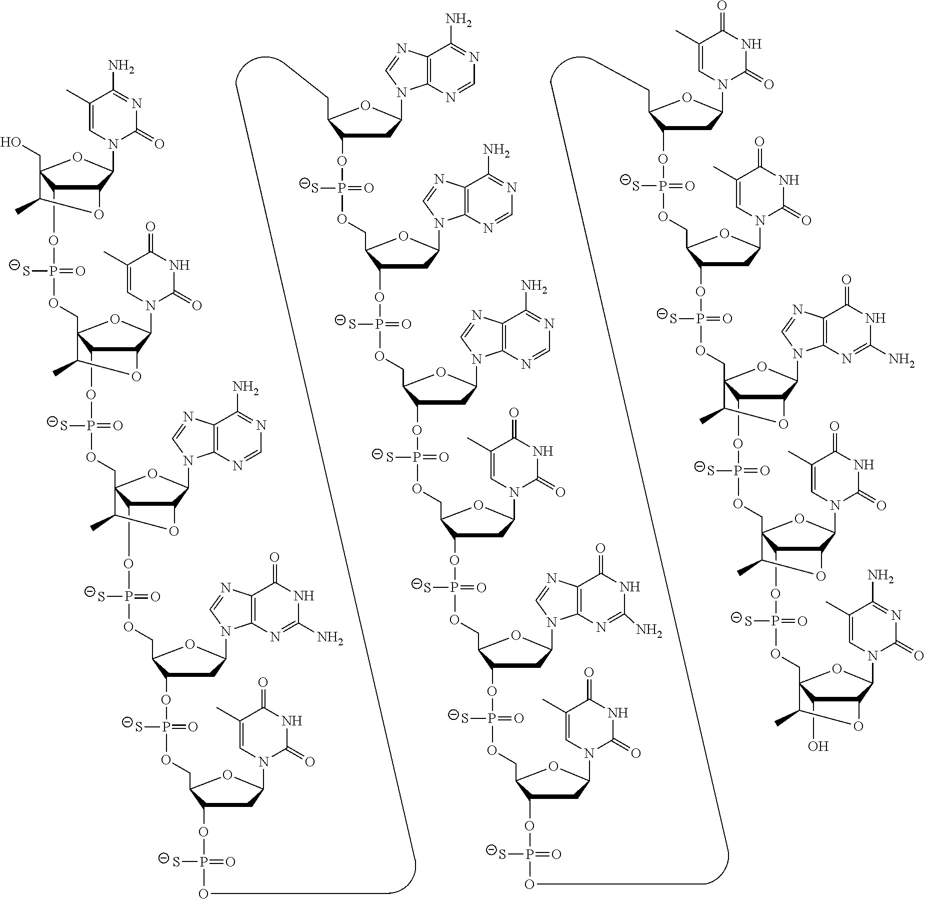

In certain embodiments, a compound comprises or consists of the sodium salt of 916602, having the following chemical structure:

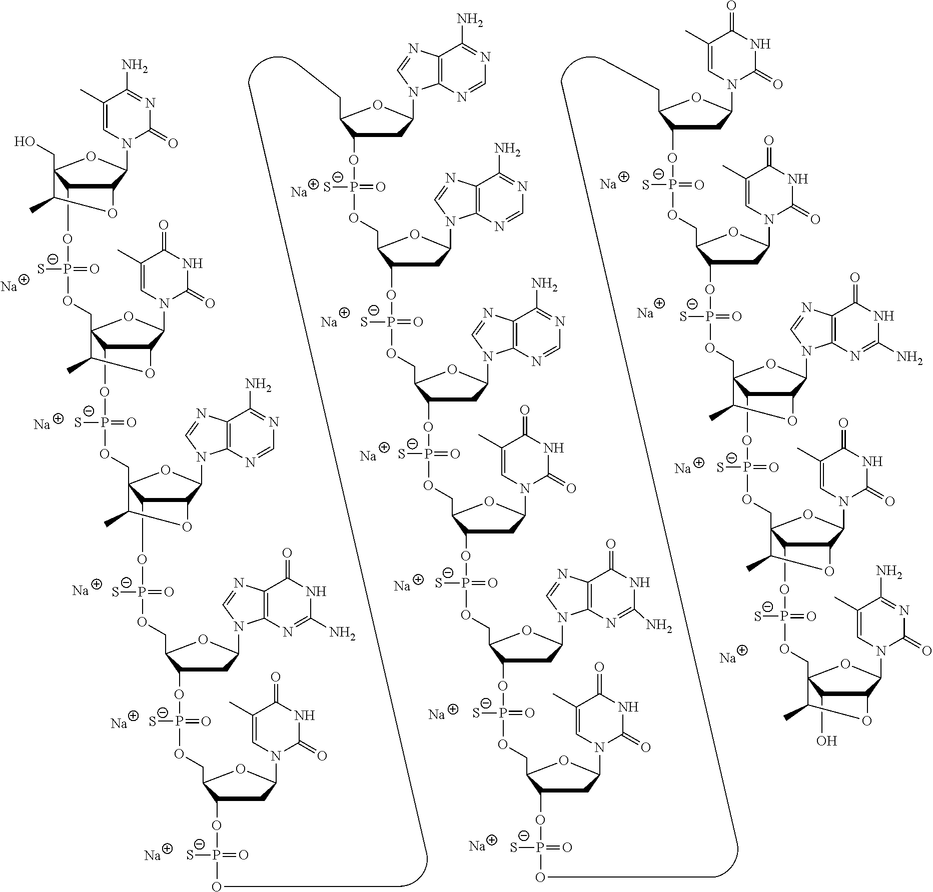

In any of the foregoing embodiments, the compound or oligonucleotide can be at least 85%, at least 90%, at least 95%, at least 98%, at least 99%, or 100% complementary to a nucleic acid encoding PNPLA3.

In certain embodiments, a compound comprises a modified oligonucleotide described herein and a conjugate group. In certain embodiments, the conjugate group is linked to the modified oligonucleotide at the 5′ end of the modified oligonucleotide. In certain embodiments, the conjugate group is linked to the modified oligonucleotide at the 3′ end of the modified oligonucleotide. In certain embodiments, the conjugate group comprises at least one N-Acetylgalactosamine (GalNAc), at least two N-Acetylgalactosamines (GalNAcs), or at least three N-Acetylgalactosamines (GalNAcs).

In certain embodiments, compounds or compositions provided herein comprise a pharmaceutically acceptable salt of the modified oligonucleotide. In certain embodiments, the salt is a sodium salt. In certain embodiments, the salt is a potassium salt.

The following examples and figures are put forth so as to provide those of ordinary skill in the art with a complete disclosure and description of how to make and use the various aspects of the invention, and are not intended to limit the scope of what the inventors regard as their invention

### FIGURE LEGEND

FIG. 1 The CE 16:1/16:0 ratio measured in both plasma and liver of wild-type and 148M knock-in mice after PNPLA3 silencing

FIG. 2 The correlation between plasma CE 16:1/16:0 and liver PNPLA3 mRNA after a single dose of PNPLA3 ASO

### EXAMPLES

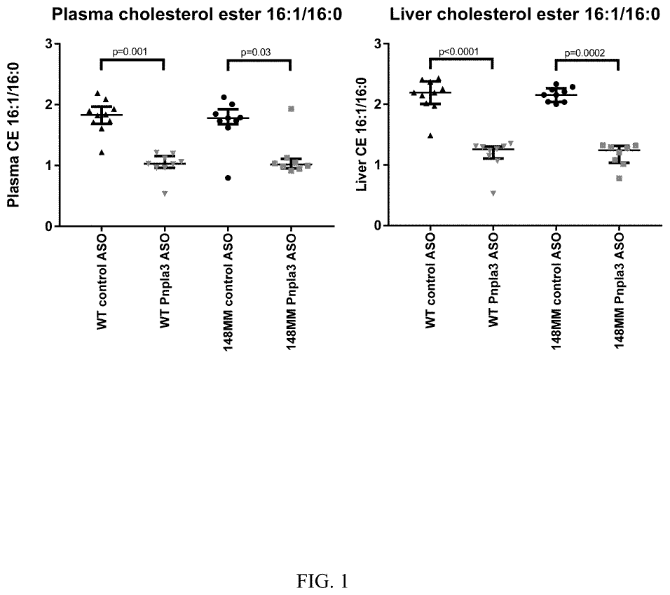

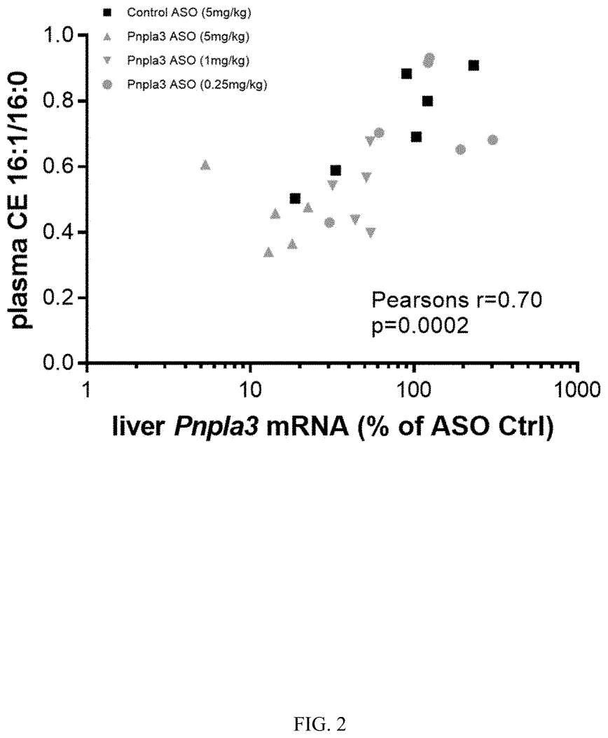

Effects of PNPLA3 silencing with a potent liver-targeted GalNAc-conjugated antisense oligonucleotide (ASO), ION 975616, in both wild-type and 148M knock-in mice, on plasma and liver lipidomics were investigated. Cohorts of mice were fed either a steatogenic high-sucrose diet or a NASH diet high in fructose, trans-fatty acids and cholesterol to induce marked liver steatosis, inflammation and fibrosis. Plasma and liver lipid composition were investigated using a combination of UPLC-MS/MS and direct infusion (shotgun). The ratio between cholesteryl palmitoleate and cholesteryl palmitate (CE 16:1/16:0) was reduced in both plasma and liver after PNPLA3 silencing (FIG. 1). This effect was independent of PNPLA3 genotype or diet. In addition, there were a correlation between plasma CE 16:1/16:0 and liver PNPLA3 mRNA after a single dose of PNPLA3 ASO (FIG. 2). The ratio between cholesteryl palmitoleate and cholesteryl palmitate (CE 16:1/16:0) in human plasma or serum can be measured by mass spectrometric detection after separation using either liquid or gas chromatography.

1. A method for the screening and/or diagnosis of a disease associated with PNPLA3 comprising measuring the CE 16:1/16:0 ratio in a patient in need of therapy of a disease associated with PNPLA3.

2. The method of claim 1, wherein the disease is chosen from liver damage, steatosis, liver fibrosis, liver inflammation, liver scarring or cirrhosis, liver failure, liver enlargement, elevated transanimases, or hepatic fat accumulation in a patient having, or at risk of having, a liver disease associated with PNPLA3.

3. The method of claim 1, wherein the disease is NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis.

4. The method of claim 1, wherein the patient in need thereof has a CE 16:1/16:0 ratio above 0.3.

5. The method of claim 4, wherein the patient in need thereof is administered a compound targeting PNPLA3 nucleic acid.

6. The method of claim 5, wherein the compound targeting PNPLA3 nucleic acid is chosen from ION 916333, ION 975616, ION 975613, ION975612, ION 916789, 916602, or salts thereof.

7. The method of claim 6, wherein the compound targeting PNPLA3 nucleic acid is chosen from ION 916333, ION 975616, or salts thereof.

8. A method of treating a disease associated with PNPLA3 in a patient in need thereof, comprising:
a) identifying a patient with a CE 16:1/16:0 ratio above 0.3; and
b) administering a compound targeting PNPLA3 nucleic acid to the patient.

9. The method of claim 8, wherein the disease is chosen from liver damage, steatosis, liver fibrosis, liver inflammation, liver scarring or cirrhosis, liver failure, liver enlargement, elevated transanimases, or hepatic fat accumulation in a patient having, or at risk of having, a liver disease associated with PNPLA3.

10. The method of claim 8, wherein the disease is NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis.

11. The method of claim 8, wherein the compound targeting PNPLA3 nucleic acid is chosen from ION 916333, ION 975616, ION 975613, ION975612, ION 916789, 916602, or salts thereof.

12. The method of claim 11, wherein the compound targeting PNPLA3 nucleic acid is chosen from ION 916333, ION 975616, or salts thereof.
